(19) United States
(12) Patent Application Publication
(10) Pub. No.: US 2003/0211505 A1
(43) Pub. Date: Nov. 13, 2003

(54) In vivo assay for identifying nucelic acid sequences involved in alveolar loss, development and nucleic acid sequences identifield thereby

(76) Inventors: Donald Massaro, Washington, DC (US); Gloria De Carlo Massaro, Washington, DC (US); Wai-Yee Chan, Potomac, MD (US)

Correspondence Address: PILLSBURY WINTHROP, LLP, P.O. BOX 10500, MCLEAN, VA, 22102, US

(21) Appl. No.: 10/275,156

(22) PCT Filed: Apr. 27, 2001
(86) PCT No.: PCT/US01/13442

(51) Int. Cl. C12Q 1/68

(52) U.S. Cl. 435/6

(57) ABSTRACT

A novel assay system for identifying nucleic acid sequences polypeptides involved in alveolar function, development and turnover is provided. These nucleic acid sequences and polypeptides have application as therapeutics for pulmonary diseases and conditions, and in screening methods for identifying compounds that “turn on” or “turn off” these nucleic acid sequences.

### CROSS REFERENCE TO RELATED APPLICATION

This application claims priority from U.S. Provisional Application Serial No. 60/200,870, filed May 1, 2000, and is incorporated herein in its entirety by reference.

### FIELD OF THE INVENTION

An in vivo assay method for identifying nucleic acid sequences, and proteins involved in alveolar turnover, i.e., loss and/or regeneration of alveoli, is provided. Nucleic acid sequences and proteins identified by this assay have potential in treating conditions involving inadequate alveoli number, function, and/or development, e.g., emphysema or premature infants having inadequate lung development. Also, the identified nucleic acid sequences and proteins themselves have application in screens for identifying compounds, e.g., small molecules, that modulate alveolar turnover, including alveolar development, function and repair, and as probes for identifying related molecules, in particular human counterpart sequences.

### BACKGROUND OF THE INVENTION

In large part because of the millions of smokers in the United States and worldwide, emphysema is a significant health problem that results in the destruction of the lung's gas-exchange structures (alveoli), and leads to inadequate oxygenation, disability and, frequently, death. Lung transplantation currently provides the only effective means of remediation. Consequently, there is a significant need in the art for identification of compounds and methods for effective treatment of emphysema.

Toward that end, several animal models have been developed wherein the animals develop functional and anatomical characteristics similar to emphysema in human subjects. Perhaps the best known animal model for the study of emphysema is the enzyme (elastase)-induced emphysema model. For example, rodents with enzyme-induced emphysema have been used in studies of the effect of inhaled intoxicants on pulmonary-impaired populations of animals.

However, several other experimental models of emphysema have been reported. (Karlinsky et al, Am. Rev. Dis., 117:1109-1133 (1978)). Some of these models have been structurally, functionally, and biochemically characterized to determine their similarities to human disease. It has specifically been reported that starvation has an effect on lung mechanics, ultra-structure and connective tissue levels. (Sahebjami et al, Am. Rev. Respir. Dis. 119:443-451 (1979); Sahebjami et al, Amer. Rev. Resp. Dis. 117:77-83 (1978); Karlinsky et al Amer. J. Physiol 251:R282-R288 (1986); Sahebjami et al, Amer. Rev. Respir. Dis.124:619-624 (1981); Sahebjami et al, J. Appl. Physiol. 58(3):778-784 (1985); Sahebjami et al, J. Appl. Physiol. 73(6):2349-2354 (1992); Sahebjami, Amer. Rev. Respir. 133:769-772 (1986); and Sahebjami et al, Amer. Rev. Respir. Dis. 126:483-487 (1982). One researcher in particular, Hamid Sahebjami, has published many reports in this area. In many of the above-referenced articles, experiments are reported which suggest that prolonged food deprivation results in some changes to the structure and function of the lung that resemble emphysematous lungs. For example, the terminal air spaces in starved lungs become grossly enlarged. (Sahebjami et al, Am. Rev. Respir. Dis. 114:443-451 (1979)). Also, ultra-structurally, enlargement of air spaces and irregularity and effacement of the inter-alveolar septa are present in starved lungs (Sahebjami et al, Am Rev. Respir. Dis. 124:619-624 (1981)). It has been hypothesized that increased surface forces found in starved lungs are related to inadequate available sources of surfactant that is replenishible with refeeding, leading to return of surface forces to control levels. (Sahebjami et al, Am. Rev. Respir. Dis. 126:483-487 (1982)). However, some studies that suggest that refeeding of starved rodents leads to restoration of surface forces without the return of tissue elasticity to normal and to less severe air-space enlargement. (Sahebjami et al, Amer. Rev. Respir. Dis. 119:443-451 (1979); Sahebjami, Amer. Rev. Respir. Dis. 133:769-772 (1986); Sahebjami et al, Am. Rev. Respir. Dis. 128:644-647 (1983); and Sahebjami et al, Amer. Rev. Respir. Dis. 128:483-487 (1982)).

Related thereto, a study wherein rodents were subjected to repeated cycles of starvation and refeeding suggested that lung growth is retarded in growing rats subjected to repeated cycles of mild starvation and refeeding, as manifested by smaller lung volume and reduced alveolar surface area. It was speculated, based on these observations, because alveolar size was unchanged, that a reduced number of alveoli was most likely responsible for the observed decrease in lung volume. (Sahebjami et al, J. Appl. Physiol. 73(6):2349-2354 (1992)).

The afore-discussed studies were made predominantly in order to study the role that nutrition may play on respiratory system function and development in both juvenile and adult animals. However, several of these articles have suggested that the starvation animal model may be a useful model for study of emphysema and potential treatments, similar to the elastase model of emphysema.

In this regard, the starvation and enzyme (elastase) models of emphysema in the rat have been compared by some researchers. (Harkema et al, Am. Rev. Respir. Dis. 129:584-591 (1984)). Also, the effects of starvation and starvation and refeeding on elastase-induced emphysema have been studied. (Sahebjami et al, J. Appl. Physiol. 66(6):2611-26126 (1989); Sahebjami et al, J. Appl. Physiol. Respir. Environ. Exercise Physiol. 48(2):284-288 (1980)).

However, the use of this animal model for the study of emphysema has been criticized by some researchers. For example, Harkema et al (Am. Rev. Respir. Dis. 129:584-591 (1984)) states at page 590 the following: “The starved rat does not appear to be a useful animal model of human pulmonary emphysema for studies of the effects of inhaled materials”, and “The alterations of in vivo pulmonary function tests in starved rats do not resemble those in enzyme-induced emphysematous rodents nor those reported in human patients with panacinar emphysema.”

Further, Sahebjami et al, Am. Rev. Respir. Dis. 124:619-624 (1981), states in his conclusion that “[l]ong-term studies are necessary to test the relation between the state of nutrition and lung function in patients with emphysema” (Sahebjami et al (Id.), at page 624)). Similarly, Sahebjami et al, J. Appl. Physiol. 73(6):234902354 (1992), refer to the “differences between the [accepted] experimental models of emphysema and the starved lung model” and, therefore, refer to this animal model as “emphysema-like” or “nutritional emphysema.” Thus, while starvation has been reported in rodents to result in a condition resembling emphysema in some of its symptoms, there are noteworthy differences, including especially loss of connective tissue components seen in the starvation model. (Sahebjami et al, J. Appl. Physiol. 58(3):778-789 (1985)).

More significantly, no researcher has suggested using this animal model for identification of nucleic acid sequences or proteins, the expression or absence of expression, modulates alveolar turnover (regeneration), including e.g., alveolar development, function and repair. Rather, as noted supra, the bulk of previous research relating to the starvation model has been focused toward determining the effects of nutrient restriction on respiratory function, both transient and permanent, in young and old animals.

### OBJECTS OF THE INVENTION

Therefore, it is an object of the invention to provide novel applications for the rodent starvation model, wherein this animal model is used to identify molecules that regulate alveolar turnover (regeneration) including, e.g., alveolar function, development and repair.

More specifically, it is an object of the invention to identify nucleic acid sequences that modulate alveolar turnover (regeneration), including e.g., alveolar function, development and repair, which sequences are expressed or not expressed during starvation, but which conversely are not expressed, or expressed during adequate nutrition, or when adequate nutrition is resumed (“refeeding”).

Also, it is an object of the invention to identify nucleic acid sequences that are expressed or not expressed upon refeeding but which are not expressed or expressed when the animal is nutritionally deprived that are involved in alveolar turnover, including, e.g., alveolar function, development and repair.

It is another object of the invention to utilize these identified nucleic acid sequences to construct probes or primers to identify related sequences in other animals, in particular humans.

It is another object of the invention to identify proteins or polypeptides that modulate alveolar turnover (regeneration) including, e.g., function, development and/or repair which are expressed or not expressed during starvation, but which conversely are not expressed or expressed during adequate nutrition and when adequate nutrition is reinstituted (“refeeding”).

It is another object of the invention to identify proteins or polypeptides that modulate alveolar turnover (regeneration) including, e.g., alveolar function, development, repair and turnover that are expressed when an animal is exposed to adequate nutrition, e.g., refeeding, but which are not expressed when the animal is nutritionally deprived (“starved”).

It is still another object of the invention to utilize the identified nucleic acid sequences, especially the regulatory portions thereof, to identify molecules that “turn-on” or “turn-off” such sequences. These molecules may have potential as in vivo agonists or antagonists in the treatment of pulmonary disorders involving inadequate or aberrant alveolar function, development, number, repair and alveolar turnover.

It is still another object of the invention to provide pharmaceutical compositions for treatment of pulmonary disorders such as emphysema that comprise proteins, nucleic acid sequences, or molecules identified by the methods described herein.

Also, it is an object of the invention to utilize the identified nucleic acid sequences in gene mapping studies to identify the chromosomal loci which comprise other genes involved in pulmonary turnover (regeneration) including, e.g., alveolar development, function, and repair. This analysis will be facilitated based on the recent report by Celera Genomics Corporation of the elucidation of the entire structure of the genome of a human individual.

### BRIEF DESCRIPTION OF THE INVENTION

To the best of the inventors' knowledge, no one has ever previously linked the use of caloric restriction and refeeding to the turnover (i.e., loss and regeneration) of alveoli or to the turning on or off of specific genes that are responsible for these changes, or further to the use of this information to obtain related or full length sequences, and polypeptides that are involved in alveolar turnover including, e.g., function, development and repair.

Rather, while previous papers discussed supra have suggested that animals that are nutritionally deprived have lungs with enlarged gas-exchange units (alveoli) and diminished alveolar surface area, these changes, which are referred to a starvation-induced emphysema, have been made largely to study the effect of nutrition on respiratory function.

The present invention hinges on the recognition that food restriction and refeeding can be used as a model for the study of respectively the physiological endogenous loss and regeneration of pulmonary alveoli that on examination of lung gene expression during food deprivation and refeeding will lead to the identification of genes responsible for the loss of alveoli in disease, for example that occur with aging and for the regeneration of alveoli; and that this information can be used for therapeutic, diagnostic and assay purposes. In particular, the identified sequences will be useful in identifying molecules, i.e., potential drug candidates that “turn on” or “turn off” these sequences. Such molecules will have potential application in treating pulmonary diseases and conditions, e.g., emphysema, bronchopulmonary dysplasia, age-related alveolar dysfunction, inadequate alveolar function in premature infants, that are characterized by inadequate alveolar numbers, development or function.

Therefore, in a first embodiment, the invention is directed toward the identification of nucleic acid sequences that modulate alveolar turnover, the expression or lack of expression is involved in the formation or loss of alveoli and/or alveoli function.

In general, this will comprise comparison of nucleic acid sequences that are expressed differently in lung tissue derived from animals that are nutritionally deprived relative to lung tissue obtained from animals that are exposed to adequate nutrition, e.g., during refeeding after previous starvation.

Methods for identifying nucleic acid sequences that are expressed in cells or tissues only under specific conditions are well known in the art and include, by way of example, subtractive hybridization, gene chip analysis, serial analysis of gene expression, and differential display methods. In fact, techniques designed to identify genes that are differentially regulated by cells under various physiological or experimental conditions (e.g., differentiation, carcinogenesis, pharmacologic treatment) have become pivotal to modem biology. Differential display is one of the latest additions to the repertoire of such techniques. This technique was introduced by Liang and Pardee and designed in U.S. Pat. No. 5,262,311. Prior to Liang and Pardee's introduction of this technique, those interested in identifying differentially expressed genes were compelled to resort either to differential hybridization screening (Zimmerman et al, Cell, Vol. 21, pp. 709-715 (1980)), or to subtractive hybridization screening (St. John et al, Cell, Vol. 16, pp. 443-452 (1979)) of complementary deoxynucleic acid (“cDNA”) libraries. Neither of these methods is entirely satisfactory; both are time consuming and labor intensive. Of the two, differential hybridization (also known as +/− screening) is more insensitive, and is therefore typically only used to detect relatively large differences between high to moderate-abundance transcripts. Subtractive hybridization, is more sensitive than +/− screening, but is technically more demanding. Also, it is necessary to carry out two separate subtractive hybridization experiments in order to identify both up- and down-regulated gene expression.

Differential display offers an attractive alternative to differential and subtractive hybridization screening. Generally, Liang et al describes a protocol which involves the reverse transcription of a messenger ribonucleic acid (“mRNA”) population, in independent reactions, with each of twelve anchor primers (T.sub. 12 MN), where M can be G (guanine), A (adenine) or C (cytosine) and N can be G, A, C or T (thymidine). The resulting single-stranded cDNAs are then amplified by the polymerase chain reaction (hereinafter “PCR”) using the same anchor primer used for reverse transcription together with an upstream or 5′ decamer of arbitrary sequence. The PCR products, which are labeled by incorporation of tracer amounts of a radioactive nucleotide, are resolved for analysis by denaturating polyacrylamide gel electrophoresis (PAGE). This technique permits the visualization of both up- and down-regulated gene expression simultaneously in the same experiment. Liang et al postulated that each two-primer combination could amplify only a limited subpopulation of cDNAs, and that the twelve anchor primers together with twenty arbitrary decamers (i.e., 240 PCR reactions) should result in the display of the 3′ termini of all distinct mRNAs that are theoretically expressed in any given cell type (Liang and Pardee, Science, Vol. 257, pp. 967-971 (1992)).

Recently, Liang and Pardee disclose in U.S. Pat. No. 5,599,672, incorporated by reference herein, a method for isolating mRNAs as cDNAs employing a polymerase amplification method using at least two oligodeoxynucleotide primers. In one approach, the first primer contains a sequence capable of hybridizing to a site immediately upstream of the first A ribonucleotide of the mRNA's poly A tail and the second primer contains an arbitrary sequence. In another approach, the first primer contains a sequence capable of hybridizing to a site including the mRNA's poly A signal sequence and the second primer contains an arbitrary sequence. The '672 patent mentions the use of three or more nucleotides that can hybridize to an mRNA sequence that is immediately upstream of the poly A tail, however, it states that using such a first primer is not practical for rapid screening of the mRNAs contained within a given cell line due to the number of oligodeoxynucleotides required to identify every mRNA.

Still further, Villeponteau et al, in U.S. Pat. No. 5,580,726, incorporated by reference herein, describes a method for detecting and isolating differentially expressed mRNAs using first oligonucleotide primers for reverse transcription of mRNAs and both the first oligonucleotide primers and second oligonucleotide primers for replication of the resultant cDNAs. These primers have a length of at least twenty-one (21) nucleotides. Furthermore, Villeponteau et al direct their claims towards the cycling parameters of the PCR reaction. Although this method may provide additional information in screening the mRNA's, as suggested by Liang and Pardee, we would expect that it would still result in a possibly significant number of false positives due to the presence of artifacts.

Another method for performing differential display that can screen differences in gene expression between various cell types or between cell in different stages of development or cells under different pharmacological conditions is disclosed in U.S. Pat. No. 6,045,998, incorporated by reference herein. The technique is reported to be highly reproducible and results in significantly lower numbers of false positives than previously reported techniques. This method uses the polymerase chain reaction (PCR) to amplify cDNA produced from a selected set of expressed mRNA sequences from particular cell types. The method includes the following steps:

(1) reverse transcription of sample mRNAs using oligo(dT) primer having from about 12 to about 18 T nucleotides to produce cDNA;

(2) quantification of the cDNA resulting from reverse transcription of step (1) to determine the amount of cDNA produced;

(3) performing a polymerase chain reaction, said performance comprising:

(a) titration of the cDNA, by running at least two different concentrations of the cDNA through the PCR; and

(b) simultaneously with step (a), adding at least one anchor primer having the sequence T.sub. 12 MNN, wherein M is A, G or C, and Nis A, G, C or T;

(4) removing cDNA from the resulting gel and reamplifying said cDNA; and

(5) subtracting contaminating cDNAs from the reamplified product.

Thus, nucleic acid sequences that are selectively expressed and not expressed upon refeeding can be identified by any of the above-described techniques or other known methods for identifying nucleic acid sequences expressed by cells only under certain conditions.

Once nucleic acid sequences have been identified which are expressed or not expressed in lung tissue or cells obtained from a rodent that is released from nutritional deprivation (“refeeding”), and which are not expressed or expressed under starvation conditions, these nucleic acid sequences will be sequenced by known methods.

The resultant sequences will preferably be used as probes to identify related sequences from human cDNA or genomic libraries, i.e., by hybridization assay or by review of previously reported human nucleic acid sequences and in particular the human genome reported by Celera. It is anticipated that these sequences will have related human counterparts that encode proteins involved in alveolar function, repair and/or development.

Also, these sequences and their human or other mammalian counterparts will be used to express the corresponding polypeptides or proteins. Such proteins or polypeptides have potential application as therapeutic agents, e.g., for treatment of emphysema, bronchopulmonary dysplasia or other disorders characterized by inadequate alveolar function, number or repair.

Another application of nucleic acid sequences identified by the subject methods involves drug screening studies. Specifically, once nucleic acid sequences have been elucidated that are “turned on” or “turned off” upon refeeding that are involved in alveolar turnover, repair, regeneration and/or function, the corresponding regulatory sequences can be used to identify compounds that turn on or off these genes. In general, this will comprise inserting these sequences in a suitable vector, e.g., a mammalian vector system wherein the regulatory sequence is operably linked to a sequence that is detectable upon expression. For example, the sequence can be operably linked to the GFP (green fluorescent protein) or a peptide (e.g., FLAG®) for which an antibody that specifically binds is available.

Compounds which activate gene expression have potential application as therapeutics for treatment of pulmonary diseases characterized by alveolar dysfunction, such as emphysema. These compounds will be subjected to further studies, i.e., preclinical studies and later clinical studies, if warranted.

Additionally, the identified nucleic acid sequences have potential application for gene therapy. This will comprise insertion of the sequence in a vector suitable for use in human subjects, e.g., an adenoviral vector or other suitable vector system. The gene will be operably linked to a strong promoter and other regulatory sequences necessary for gene expression. Thereby, when the vector is introduced in a subject it will provide for expression of a protein that modulates alveolar turnover, repair, regeneration and/or function. In this embodiment of the invention, a vector will preferably be administered via an inhalatory route so that it is directly accessible to lung tissue. Methods for administering vectors, including adenoviral vectors in a manner by which they reach pulmonary sites are known. In fact, these methods have reported application in delivery of genes for treatment of cystic fibrosis. Also, the pulmonary delivery of alpha anti-trypsin for treatment of emphysema has been reported in the literature.

The vector will be in a suitable carrier, e.g., a liposomal delivery system. Desirably, the vector will be administered in conjunction with an immunosuppressant in order to reduce the possibility of the treated subject eliciting an immune response to the vector.

### EXAMPLES

Nucleic acid sequences that modulate alveolar tumor (regeneration), including repair, development, and function, are identified by a protocol that allows for comparison of nucleic acid sequences expressed by lung cell or tissues obtained under starvation relative to non-starvation conditions, i.e., after adequate nutrition has been resumed. Preferably, lung cells or tissues will be obtained from animals, e.g., rodents such as mice or rats, under starvation conditions, and an analogous lung cell or tissue sample will be obtained from an animal, preferably mouse or rat, shortly after normal nutrition has been resumed, i.e., within a day or several days after normal nutrition has been resumed. These protocols are described below.

Subtractive Hybridization

Subtractive hybridization allows one to obtain relatively small cDNA libraries enriched with transcripts that are present in only one of the compared tissues. The subtraction library is generated using the Clontech PCR-Select® cDNA Subtraction Kit (Clontech Lab, Palo Alto, Calif.). Poly A⁺RNA, 0.5-2.0 μg, prepared from lungs of rats will be used as template for ds cDNA synthesis. Double stranded cDNA, 100 ng, is digested with Rsa I, extracted with phenol, and precipitated by ethanol. The tester Rsa-digested ds cDNA is divided into two portions, and each is ligated to adaptor 1 or adaptor 2R provided in the kit. To carry out the first round of subtractive hybridization, an excess of driver ds cDNA (˜600 ng) is added to each of the two tubes containing ˜20 ng of adaptor 1- and adaptor 2-ligated tester cDNA. The samples are mixed, heat-denatured (1.5 min at 98° C.) and allowed to anneal for ten hours at 68° C. After this first hybridization, the two samples are combined and a fresh portion of heat-denatured driver (˜150 ng) is added. The sample is allowed to hybridize for an additional ten hours at 68° C. The entire population of cDNAs is then subjected to two rounds of PCR. The primary PCR is carried out using primer P1 and P2R. Some of the amplified products are then used as a template in secondary PCCR for ten cycles under the same conditions used for the primary PCR, except PCR primer P1 and P2 are replaced with nested PCR primer PN1 and PN2R. Products from the secondary PCRs are inserted into pCR®II using the TA-cloning kit (Invitrogen Corp., San Diego, Calif.), as described earlier. This is the subtractive cDNA library. The same procedure will be repeated. However, this time after secondary PCR, the enriched subtracted cDNAs will be used as hybridization probes to screen the subtractive cDNA library previously prepared. This approach allows amplification of target differentially expressed cDNAs, eliminates any intermediate steps for physical separation of ss and ds cDNA, and simultaneously suppresses amplification of non-target DNA (Diatchenko et al., Proc Natl Acad Sci USA, 93:6025 (1996); Gurskaya et al, Anal Biochem. 240:90 (1996)). After confirmation of the differential expression of the positive clones with Northern blot analysis, Ribonuclease Protection Assay or polymerase chain reaction, the nucleotide sequence of the cDNA insert will be determined by the dideoxy chain termination method. Full-length cDNA will be obtained by screening an appropriate regular cDNA library using the cloned insert as probe.

Serial Analysis of Gene Expression

Serial Analysis of Gene Expression (SAGE) was designated to generate short tags for all expressed sequences (known or unknown) in a cell or tissue using a relatively simple procedure [Velculescu VE et al., Science, 270(1995)484]. SAGE is based on two basic principles: 1) when isolated from a defined position in a transcript, e.g., a restriction enzyme site, a tag (short nucleotide sequence of 9 to 10 bp) can uniquely identify a transcript; 2) concatenation of tags in a serial manner allows the efficient analysis of transcripts by the sequencing of multiple tags [Velculescu VE et al., Science, 270 (1995) 484]. SAGE is based on two basic principles: 1) when isolated from a defined position in a transcript, e.g., a restriction enzyme site, a tag (short nucleotide sequence of 9 to 10 bp) can uniquely identify a transcript; 2) concatenation of tags in a serial manner allows the efficient analysis of transcripts by the sequencing of multiple tags within a single clone. Poly(A) mRNA will be isolated from cells or tissues using the TRIzol Reagent (GIBCO-BRL Life Technologies, Rockville, Md.), the Rneasy Total RNA Isolation Kit, and Oligotex Direct mRNA Kit (QIAGEN, Valencia, Calif.). The SuperScript Choice System (GIBCO-BRL Life Technologies, Rockville, Md.) will be used for the synthesis of ds cDNA except that a 5′-biotinylated oligo-(dT)₁₈ (Genosys Biotechnologies, Woodlands, Tex.) will be used as primer for reverse transcription in the synthesis of the first strand. The biotinylated cDNAs will then be cleaved with Nla III (anchoring enzyme) (NEB, Beverly, Mass.). The poly(A) containing portion of the cleaved cDNA will be isolated by binding to Dynabeads M-280 Streptavidin (Dynal, Lake Success, N.Y.). The bound cDNA will be divided in half and ligated via the anchoring restriction site to one of two linkers (Genosys Biotechnologies, Woodlands, Tex.) containing the recognition site for the tagging enzyme BsmF1. After ligation, the beads will be washed and the SAGE tags released from both pools by digestion with BsmF1 (NEB, Beverly, Mass.). After blunt ends are created at the 3′ end of the tag, the two pools of released tags will be ligated to each other. Ligated tags then serve as templates for PCR amplification with primers specific to each linker. The amplified 102 bp products contain two tags (one di-tag) linked tail to tail and flanked by sites for the anchoring enzyme. Cleavage of the PCR product with Nla III generates di-tags that will then be ligated to generate concatemers. Tag concatemers will be cloned into Sph I (NEB, Beverly, Mass.) cleaved pZero vector (Invitrogen, San Diego, Calif.). The tag concatemer-vector recombinants will be used to transform TOP 10 F′ cells (Invitrogen) to generate the SAGE library. Clones picked from the SAGE library will be sequenced using the dideoxy sequencing method. The Nla III recognition sequence provides punctuation per di-tag in the final sequence. The SAGE data generated will be analyzed using the SAGE Software version 3.04 provided to us by Dr. Kinzler (Johns Hopkins University).

Gene Chip or Microarray Hybridization Analysis

Microarray hybridization is capable of profiling gene expression patterns of tens of thousands of known genes in a single experiment. In an array experiment, many short cDNAs (cDNA microarray) or gene-specific 25-mer oligonucleotides (Oligo microarray) are individually arrayed on a single matrix. This matrix is then simultaneously probed with fluorescently tagged complementary antisense RNA (cRNA) representations of total RNA pools from test and reference cells or tissues, allowing one to determine the relative amount of transcript present in the pool by the fluorescent signal generated. Relative message abundance is inherently based on a direct comparison between a test cell/tissue state and a reference cell/tissue state (Duggan et al., Nat Genet. 21 (Supp) (1999)10; Lipshutz et al., Nat Genet. 21 (Suppl)(1999)20]. Biotin-labeled cRNA used to hybridize to the GeneChips will be prepared according to the Affymetrix GeneChip Expression Analysis Technical Manual (Research Genetics, Huntsville, Ala.). Preparation of poly(A) mRNA and synthesis of ds cDNA will be the same as described for the SAGE procedure except that the reverse transcription primer is a T7-(dT)₂₄ supplied by Research Genetics, Inc. Biotin-labeled antisense cRNA will be generated by in vitro transcription using the ENZO BioArray High Yield RNA Transcript Labeling Kit from Affymetrix. Hybridization of the cRNA probe to the GeneChip and analysis of data generated will be done by Research Genetics, Inc. Mouse GeneChip arrays manufactured by Affymetrix (Santa Clara, Calif.) will be used to compare the profile of gene expression in lung tissues of mice.

What is claimed is:

1. An assay method for identifying nucleic acid sequences or polypeptides that regulate at least one of alveolar turnover, development, function and/or repair, comprising the following steps:
(i) subjecting a non-human mammal to nutrient deprivation conditions that result in said mammal exhibiting alveolar abnormalities and dysfunction;
(ii) obtaining a lung tissue or cell sample from said mammal while nutrient deprivation conditions and alveolar abnormalities are present;
(iii) thereafter subjecting said non-human mammal to adequate nutrition (“refeeding”) in order to at least partially restore said alveolar abnormalities and/or dysfunction;
(iv) obtaining at least one lung tissue or cell sample from said animal after adequate nutrition (“refeeding”) has been reinstituted; and
(v) comparing the nucleic sequences that are present in the cell sample of (ii) and (iv) in order to identify nucleic acid sequences that are putatively involved in regulation of at least one of alveolar turnover, development, function, and repair.

2. The assay method of claim 1, wherein the comparison step (v) is effected by at least one method selected from the group consisting of subtractive hybridization, differential hybridization, gene chip analysis, serial analysis of gene expression, and differential display.

3. The assay method of claim 1, wherein the comparison step (v) is effected by differential display.

4. The assay method of claim 3, which further comprises sequencing nucleic acid sequences that are only present in cell sample (iv).

5. The assay method of claim 4, which further comprises constructing probes corresponding to said sequences, and using said probes to screen a human cDNA, or genomic library.

6. The assay method of claim 5, wherein said sequences are compared to the sequenced human genome in order to identify nucleic acid sequences that are at least 75% homologous thereto (based on sequence identity).

7. The assay method of claim 5, wherein said sequences are compared to the sequenced human genome to identify nucleic acid sequences that are at least 90% homologous thereto (based on sequence identity).

8. A method for identifying compounds that regulate at least one of alveolar turnover, function, development, and repair, comprising obtaining a regulatory (promoter) sequence from a nucleic acid sequence identified according to claim 1, operably linking said regulatory sequence to a DNA encoding a detectable protein, introducing said operably linked regulatory sequence and DNA encoding a detectable protein in a cell that provides for the expression thereof, and contacting said cell with compounds that are being screened for their potential ability to modulate alveolar turnover, function, report or regeneration, and selecting compounds that modulate at least one of alveolar turnover, repair and regeneration function based on their ability to “turn on” or “turn off” said regulatory sequence as evidenced by expression or absence of expression of the detectable protein.

9. The method of claim 8, wherein said detectable protein is GFP.

10. The method of claim 8, wherein said tested compound is a retinoid compound.

11. The method of claim 1, wherein said cell also expresses the gene product normally expressed by said regulatory sequence.

12. The method of claim 1, wherein said identified nucleic acid sequence or sequences are used to identify the corresponding human chromosomal locus or loci that comprise genes involved in alveolar turnover, function, development, and repair.

13. A nucleic acid sequence identified by the method of claim 1.

14. A polypeptide encoded by said nucleic acid sequence.
